(19) United States
(12) Patent Application Publication
(10) Pub. No.: US 2021/0159651 A1
(43) Pub. Date: May 27, 2021

(54) ELECTRONIC DEVICE INCLUDING BIOMETRIC SENSOR AND ELECTRODE FOR ACQUIRING BIOMETRIC SIGNAL

(71) Applicant: SAMSUNG ELECTRONICS CO., LTD., Suwon-si (KR)

(72) Inventors: Jaejong RYU, Suwon-si (KR); Myeounghyeoun DO, Suwon-si (KR); Hyohoon SHIN, Suwon-si (KR)

(21) Appl. No.: 16/953,881

(22) Filed: Nov. 20, 2020

(30) Foreign Application Priority Data
Nov. 22, 2019 (KR) 10-2019-0151632

(51) Int. Cl. H01R 35/04; A61B 5/0408

(52) U.S. Cl. CPC H01R 35/04; A61B 5/0408; A61B 2562/227; H01R 2201/12; H01R 2201/20

(57) ABSTRACT

An electronic device includes: a housing having at least one through-hole in a surface thereof, a rotatable wheel coupled to the surface of the housing and including a conductive portion, a printed circuit board disposed in the housing, and a first bearing structure comprising a bearing, at least part of which is disposed in the through-hole to support rotation of the wheel member, wherein the first bearing structure electrically connects the printed circuit board and the conductive portion.

### CROSS-REFERENCE TO RELATED APPLICATION

This application is based on and claims priority under 35 U.S.C. § 119 to Korean Patent Application No. 10-2019-0151632, filed on Nov. 22, 2019, in the Korean Intellectual Property Office, the disclosure of which is incorporated by reference herein its entirety.

### BACKGROUND

### 1. Field

The disclosure relates to an electronic device including a biometric sensor and an electrode for acquiring a biometric signal.

### 2. Description of Related Art

A wearable electronic device (hereinafter, referred to as the electronic device) may be worn on a user's wrist. The electronic device may include a biometric sensor for sensing biometric information of the user. The biometric sensor may include an electrocardiography sensor. The electronic device may measure an electrocardiogram of the user through a part of the user's body.

The electronic device requires at least two electrodes that make contact with the user's body to measure the electrocardiogram of the user. One of the electrodes may make contact with the user's wrist. Another one may be formed on a housing of the electronic device.

The electronic device may provide a wireless communication function. To this end, part of a metal portion of the housing may function as an antenna. Meanwhile, an electrode with which a part of the user's body is brought into contact may be formed on the housing. The electrode and the antenna may interfere with each other, which may deteriorate wireless communication performance and may cause a problem in the accuracy of biometric information.

The above information is presented as background information only to assist with an understanding of the disclosure. No determination has been made, and no assertion is made, as to whether any of the above might be applicable as prior art with regard to the disclosure.

### SUMMARY

Embodiments of the disclosure address at least the above-mentioned problems and/or disadvantages and to provide at least the advantages described below. Accordingly, embodiments of the disclosure provide an electronic device including an electrode provided on a surface of the electronic device without interference with an antenna. For example, embodiments of the electronic device may include an electrode formed in a separate wheel member that is coupled to a housing so as to be rotatable.

In accordance with an example embodiment of the disclosure, an electronic device includes: a housing having at least one through-hole formed in a surface thereof, a rotatable wheel coupled to the surface of the housing, the wheel including a conductive portion, a printed circuit board disposed in the housing, and a first bearing structure including a bearing, at least part of which is disposed in the through-hole to support rotation of the wheel, wherein the first bearing electrically connects the printed circuit board and the conductive portion.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the present disclosure will be more apparent from the following detailed description, taken in conjunction with the accompanying drawings, in which:

FIG. 1 is a front perspective view of an example electronic device according to various embodiments;

FIG. 2 is a rear perspective view of the electronic device according to various embodiments;

FIG. 3 is an exploded perspective view of the electronic device according to various embodiments;

FIG. 4 is a diagram illustrating an electronic device according to various embodiments;

FIG. 5 is a sectional view of the electronic device according to various embodiments;

FIGS. 6A and 6B are diagrams illustrating an example bearing structure of the electronic device according to various embodiments;

FIGS. 7A and 7B are diagrams illustrating an example bearing structure of the electronic device according to various embodiments;

FIG. 8 is a diagram illustrating an example bearing structure of the electronic device according to various embodiments;

FIGS. 9A and 9B are diagrams illustrating example bearing structures and a wheel member of the electronic device according to various embodiments; and

FIGS. 10A and 10B are block diagrams illustrating example biometric signal acquisition systems of an electronic device according to various embodiments.

### DETAILED DESCRIPTION

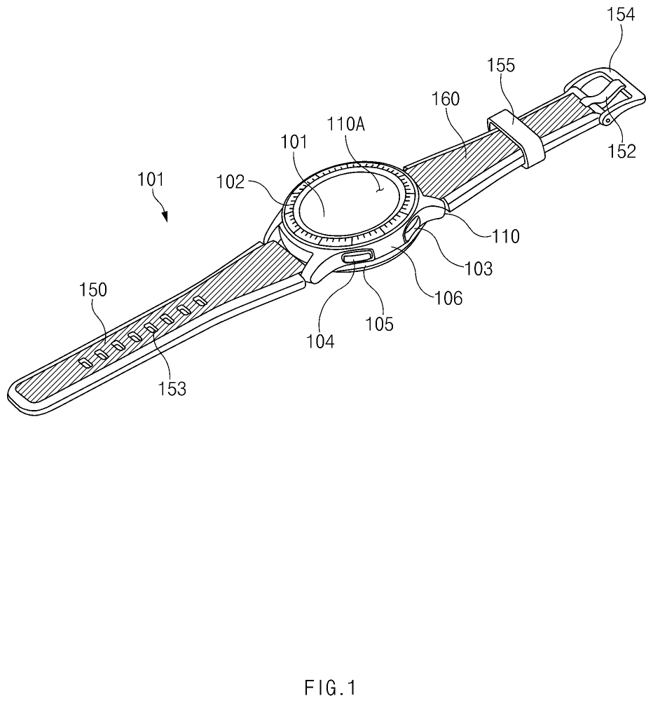

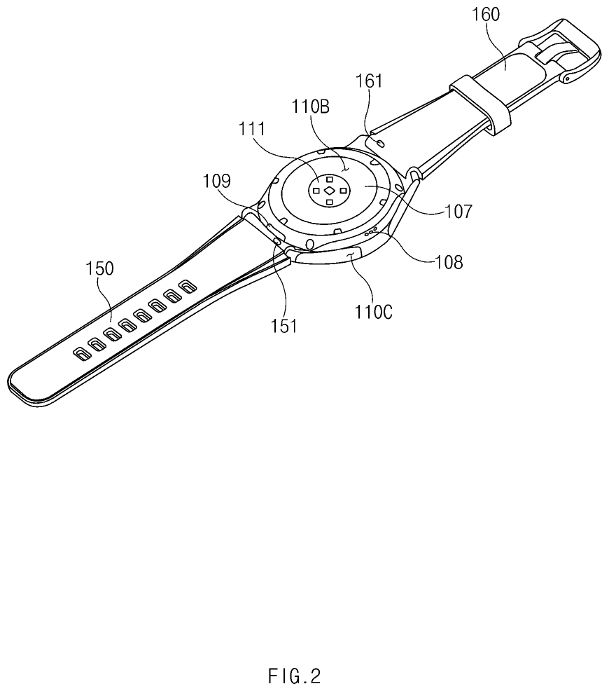

FIG. 1 is a front perspective view of an electronic device according various embodiments. FIG. 2 is a rear perspective view of the electronic device according various embodiments.

Referring to FIGS. 1 and 2, the electronic device 100 according to an embodiment may include a housing 110 that includes a first surface (or, a front surface) 110A, a second surface (or, a rear surface) 110B, and side surfaces 110C surrounding a space between the first surface 110A and the second surface 110B, and fastening members 150 and 160 (e.g., straps) connected to at least parts of the housing 110 and configured to detachably fasten the electronic device 100 on a part (e.g., a wrist, an ankle, or the like) of a user's body. In an embodiment (not illustrated), a housing may refer to a structure that forms some of the first surface 110A, the second surface 110B, and the side surfaces 110C of FIG. 1. According to an embodiment, the first surface 110A may be formed by a front plate 101, at least part of which is substantially transparent (e.g., a glass plate including various coating layers, or a polymer plate). The second surface 110B may be formed by a back plate 107 that is substantially opaque. The back plate 107 may be formed by, for example, coated or colored glass, ceramic, polymer, metal (e.g., aluminum, stainless steel (STS), or magnesium), or a combination of at least two of the aforementioned materials. The side surfaces 110C may be formed by a side bezel structure (or, a “side member”) 106 that is coupled with the front plate 101 and the back plate 107 and that contains metal and/or polymer. In some embodiments, the back plate 107 and the side bezel structure 106 may be integrally formed with each other and may contain the same material (e.g., a metallic material such as aluminum). The fastening members 150 and 160 may be formed of various materials and may have various forms. The fastening members 150 and 160 may be formed of woven fabric, leather, rubber, urethane, metal, ceramic, or a combination of at least two of the aforementioned materials and may be implemented in an integrated form or with a plurality of unit links that are movable relative to each other.

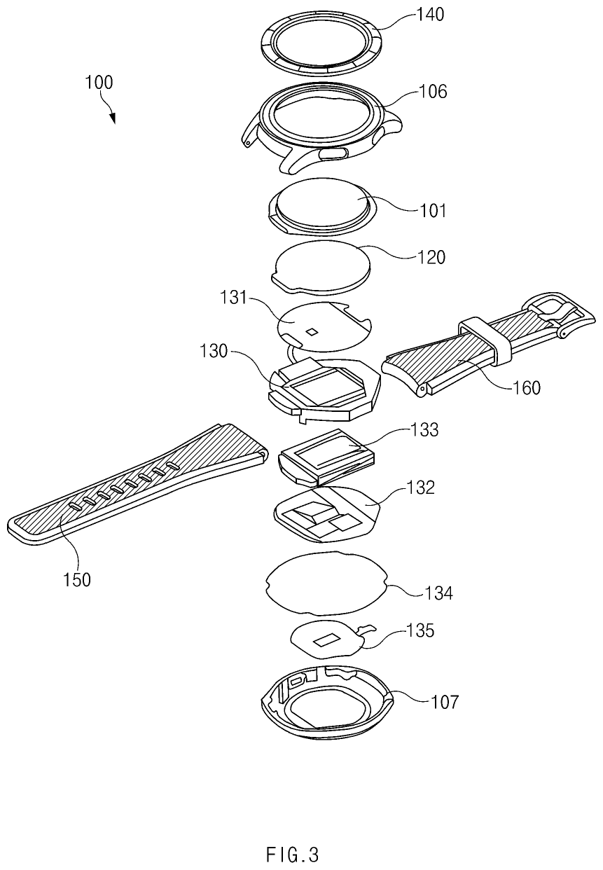

According to an embodiment, the electronic device 100 may include at least one of a display 120 (refer to FIG. 3), audio modules 105 and 108, a sensor module 111, key input devices 102, 103, and 104, and a connector hole 109. In some embodiments, the electronic device 100 may omit at least one component (e.g., the key input devices 102, 103, and 104, the connector hole 109, or the sensor module 111) among the aforementioned components, or may additionally include other component(s).

The display 120 may be visible through, for example, a large portion of the front plate 101. The display 120 may have a shape corresponding to the shape of the front plate 101. The display 120 may have various shapes such as a circular shape, an oval shape, a polygonal shape, or the like. The display 120 may be combined with, or disposed adjacent to, touch detection circuitry, a pressure sensor capable of measuring the intensity (pressure) of a touch, and/or a fingerprint sensor.

The audio modules 105 and 108 may include the microphone hole 105 and the speaker hole 108. A microphone for obtaining a sound from the outside may be disposed in the microphone hole 105. In some embodiments, a plurality of microphones may be disposed in the microphone hole 105 to detect the direction of a sound. The speaker hole 108 may be used for an external speaker and a call receiver. In some embodiments, the speaker hole 108 and the microphone hole 105 may be implemented with one hole, or a speaker (e.g., a piezo speaker) may be included without the speaker hole 108.

The sensor module 111 may generate an electrical signal or a data value that corresponds to an operational state inside the electronic device 100 or an environmental state external to the electronic device 100. The sensor module 111 may include, for example, the biometric sensor module 111 (e.g., an HRM sensor) that is disposed on the second surface 110B of the housing 110. The electronic device 100 may further include a non-illustrated sensor module, which may be, for example, at least one of a gesture sensor, a gyro sensor, an atmospheric pressure sensor, a magnetic sensor, an acceleration sensor, a grip sensor, a color sensor, an infrared (IR) sensor, a biometric sensor, a temperature sensor, a humidity sensor, or an illuminance sensor.

The key input devices 102, 103, and 104 may include the wheel key 102 disposed on the first surface 110A of the housing 110 and rotatable in at least one direction and/or the side key buttons 103 and 104 disposed on the side surfaces 110C of the housing 110. The wheel key 102 may have a shape corresponding to the shape of the front plate 101. In another embodiment, the electronic device 100 may not include all or some of the aforementioned key input devices 102, 103, and 104, and the key input devices 102, 103, and 104 not included may be implemented in different forms such as soft keys on the display 120. The connector hole 109 may include another connector hole (not illustrated) for accommodating a connector (e.g., a USB connector) that transmits and receives electric power and/or data with an external electronic device and accommodating a connector that transmits and receives audio signals with an external electronic device. The electronic device 100 may further include, for example, a connector cover (not illustrated) that covers at least part of the connector hole 109 and blocks infiltration of external foreign matter into the connector hole 109.

The fastening members 150 and 160 may be detachably fastened to at least partial areas of the housing 110 using locking members 151 and 161. The fastening members 150 and 160 may include one or more of a fixing member 152, fixing member fastening holes 153, a band guide member 154, and a band fixing ring 155.

The fixing member 152 may be configured to fix the housing 110 and the fastening members 150 and 160 to a part (e.g., a wrist, an ankle, or the like) of the user's body. The fixing member fastening holes 153 may fix the housing 110 and the fastening members 150 and 160 to the part of the user's body to correspond to the fixing member 152. The band guide member 154 may be configured to restrict a movement range of the fixing member 152 when the fixing member 152 is fastened to the fixing member fastening hole 153, thereby allowing the fastening members 150 and 160 to be brought close contact with and fastened with the part of the user's body. The band fixing ring 155 may restrict a movement range of the fastening members 150 and 160 in a state in which the fixing member 152 is fastened to the fixing member fastening hole 153.

FIG. 3 is an exploded perspective view of the electronic device according to various embodiments.

Referring to FIG. 3, the electronic device 100 may include the side bezel structure 106, a wheel key 140, the front plate 101, the display 120, a first antenna 131, a second antenna 135, a support member 130 (e.g., a bracket), a battery 133, a printed circuit board 132, a sealing member 134, the back plate 107, and the fastening members 150 and 160.

The support member 130 may be disposed inside the electronic device 100 and may be connected with the side bezel structure 106, or may be integrally formed with the side bezel structure 106. The support member 130 may be formed of, for example, a metallic material and/or a nonmetallic (e.g., polymer) material. The display 120 may be coupled to one surface of the support member 130, and the printed circuit board 132 may be coupled to an opposite surface of the support member 130. A processor, a memory, and/or an interface may be mounted on the printed circuit board 132. The processor may include, for example, one or more of a central processing unit, an application processor, a graphic processing unit (GPU), a sensor processor, or a communication processor.

The memory may include, for example, a volatile memory or a nonvolatile memory. The interface may include, for example, a high definition multimedia interface (HDMI), a universal serial bus (USB) interface, an SD card interface, and/or an audio interface. The interface, for example, may electrically or physically connect the electronic device 100 with an external electronic device and may include a USB connector, an SD card/MMC connector, or an audio connector.

The battery 133, which is a device for supplying electric power to at least one component of the electronic device 100, may include, for example, a primary cell that is not rechargeable, a secondary cell that is rechargeable, or a fuel cell. At least part of the battery 133 may be disposed on, for example, substantially the same plane as the printed circuit board 132. The battery 133 may be integrally disposed inside the electronic device 100, or may be disposed so as to be detachable from the electronic device 100.

The first antenna 131 may be disposed between the display 120 and the support member 130. The first antenna 131 may include, for example, a near field communication (NFC) antenna, a wireless charging antenna, and/or a magnetic secure transmission (MST) antenna. The first antenna 131, for example, may perform short-range communication with an external device, or may wirelessly transmit and receive electric power required for charging, and may transmit a magnetism-based signal including a short-range communication signal or payment data. In another embodiment, an antenna structure may be formed by part of the side bezel structure 106 and/or part of the support member 130, or a combination thereof.

The second antenna 135 may be disposed between the printed circuit board 132 and the back plate 107. The second antenna 135 may include, for example, a near field communication (NFC) antenna, a wireless charging antenna, and/or a magnetic secure transmission (MST) antenna. The second antenna 135, for example, may perform short-range communication with an external device, or may wirelessly transmit and receive electric power required for charging, and may transmit a magnetism-based signal including a short-range communication signal or payment data. In another embodiment, an antenna structure may be formed by part of the side bezel structure 106 and/or part of the back plate 107, or a combination thereof.

The sealing member 134 may be located between the side bezel structure 106 and the back plate 107. The sealing member 134 may be configured to block moisture and foreign matter introduced from the outside into a space surrounded by the side bezel structure 106 and the back plate 107.

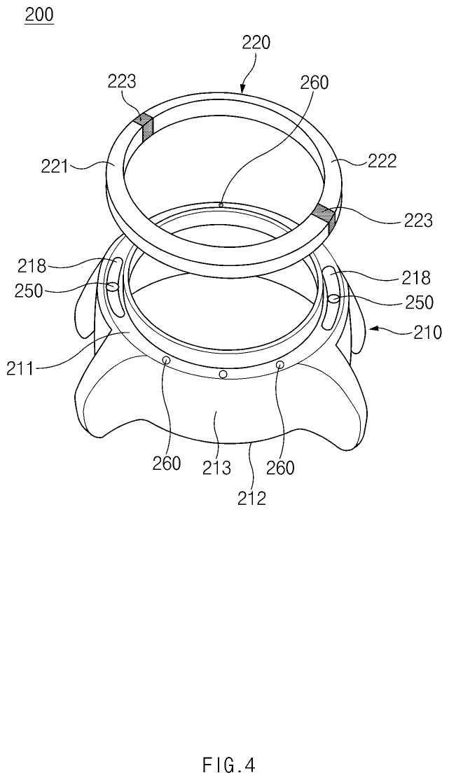

FIG. 4 is a diagram illustrating an example electronic device according to various embodiments.

In the illustrated embodiment, the electronic device 200 may include a housing 210, a wheel member (e.g., including a wheel) 220 coupled to the housing 210 so as to be rotatable, and bearing structures (e.g., including a bearing) 250 and 260 formed on the housing 210 to support rotation of the wheel member 220.

In the illustrated embodiment, the housing 210 may include a first surface 211 (e.g., the first surface 110A of FIG. 1), a second surface 212 (e.g., the second surface 110B of FIG. 2) that is opposite to the first surface 211, and side surfaces 213 (e.g., the side surfaces 110C of FIG. 1) that are formed between the first surface 211 and the second surface 212. The first surface 211, the second surface 212, and the side surfaces 213 may form an interior space of the housing 210. A printed circuit board (e.g., the printed circuit board 132 of FIG. 3), a battery (e.g., the battery 133 of FIG. 3), and a display (e.g., the display 120 of FIG. 3) may be disposed in the interior space of the housing 210. An opening may be formed in the first surface 211 of the housing 210. The display may be visible through the opening. The wheel member 220 may be seated on an area around the opening formed in the first surface 211. A part of the bearing structures 250 and 260 may be exposed on the first surface 211.

In an embodiment, the wheel member 220 may be disposed on the area around the opening formed in the first surface 211 of the housing 210. The wheel member 220 may be formed in a circular ring shape. The wheel member 220 may be configured to rotate about the central axis thereof. The central axis may be aligned with the central axis of the opening of the housing 210. Rotation of the wheel member 220 may be supported by the bearing structures 250 and 260. The wheel member 220 may include one or more conductive portions 221 and 222 and one or more insulating portions 223. The wheel member 220 may include the first conductive portion 221, the second conductive portion 222, and the insulating portions 223 for insulating the first conductive portion 221 and the second conductive portion 222. The conductive portions 221 and 222 may be electrically connected with the first bearing structures 250.

In an embodiment, at least parts of the bearing structures 250 and 260 may protrude from the area around the opening. The bearing structures 250 and 260 may include protruding portions that support rotation of the wheel member 220. The protruding portions may be implemented with parts of spheres. For example, the protruding portions may be parts of balls (e.g., bearings).

The bearing structures 250 and 260 may include the first bearing structures 250 and the second bearing structures 260. The first bearing structures 250 may be electrically connected with the conductive portions 221 and 222 of the wheel member 220 and may be configured to support rotation of the wheel member 220. Each of the first bearing structures 250 may include a conductive protruding portion (e.g., a ball or a pogo pin which may be referred to hereinafter as a bearing) and an insulating area 218 formed around the conductive protruding portion. The insulating area 218 may insulate the conductive protruding portion and the remaining area of the housing 210. The second bearing structures 260 may be configured to support rotation of the wheel member 220. Each of the second bearing structures 260 may include a non-conductive protruding portion (e.g., a ball (e.g., bearing) containing an insulating material).

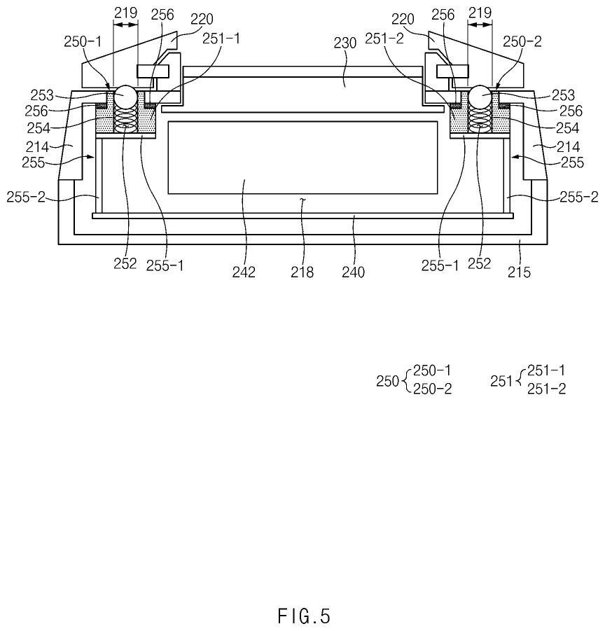

FIG. 5 is a sectional view of the electronic device according to various embodiments.

Referring to FIG. 5, the housing 210 may include a front cover 214 (e.g., the front plate 101 of FIG. 1) and a back cover 215 (e.g., the back plate 107 of FIG. 2). A battery 242 and a printed circuit board 240 may be disposed between the front cover 214 and the back cover 215. A display 230 may be disposed on part of the front cover 214. The wheel member 220 may be coupled to the front cover 214 so as to be rotatable. The first bearing structures 250 may be disposed on a peripheral area of the front cover 214.

In an embodiment, each of the first bearing structures 250 may include a bracket 251, a conductive ball 253, an elastic member 254, a waterproof member 256, and a connecting member 255.

In an embodiment, at least part of the bracket 251 may be disposed in a through-hole 219 formed in the peripheral area of the front cover 214. The bracket 251 may be formed to surround part of the conductive ball 253 and the elastic member 254.

In an embodiment, at least part of the bracket 251 may be disposed in the through-hole 219 formed in the front cover 214. The bracket 251 may include a first portion 251-1 formed in a size corresponding to the through-hole 219, a second portion 251-2 formed to be larger than the through-hole 219, and an opening 252 formed through the first portion 251-1 and the second portion 251-2. The conductive ball 253 may be disposed in the opening 252 included in the first portion 251-1, and the elastic member 254 may be disposed in the opening 252 included in the second portion 251-2. A support portion 255-1 of the connecting member 255 may be disposed on the second portion 251-2 to cover the opening 252.

In an embodiment, at least part of the conductive ball 253 may protrude from a surface of the front cover 214 through the through-hole 219. The conductive ball 253 may make contact with the elastic member 254 and a conductive portion (e.g., the conductive portion 221 or 222 of FIG. 4) of the wheel member 220. The conductive ball 253 may electrically connect the elastic member 254 and the conductive portion (e.g., the conductive portion 221 or 222 of FIG. 4) of the wheel member 220.

In an embodiment, the elastic member 254 may electrically connect the conductive ball 253 and the connecting member 255. The elastic member 254 may contain a conductive material. The elastic member 254 may apply an elastic force to the conductive ball 253 such that the conductive ball 253 makes contact with the wheel member 220.

In an embodiment, the connecting member 255 may include a flexible printed circuit board. The connecting member 255 may include the support portion 255-1 on which the elastic member 254 is disposed and an extension portion 255-2 extending from the support portion 255-1 to the printed circuit board 240. The connecting member 255 may electrically connect the elastic member 254 and the printed circuit board 240.

In an embodiment, the waterproof member 256 may be disposed between the bracket 251 and the front cover 214. The waterproof member 256 may be disposed on an area around the through-hole 219 formed in the front cover 214. The waterproof member 256 may waterproof the interior space of the housing 210 through the through-hole 219.

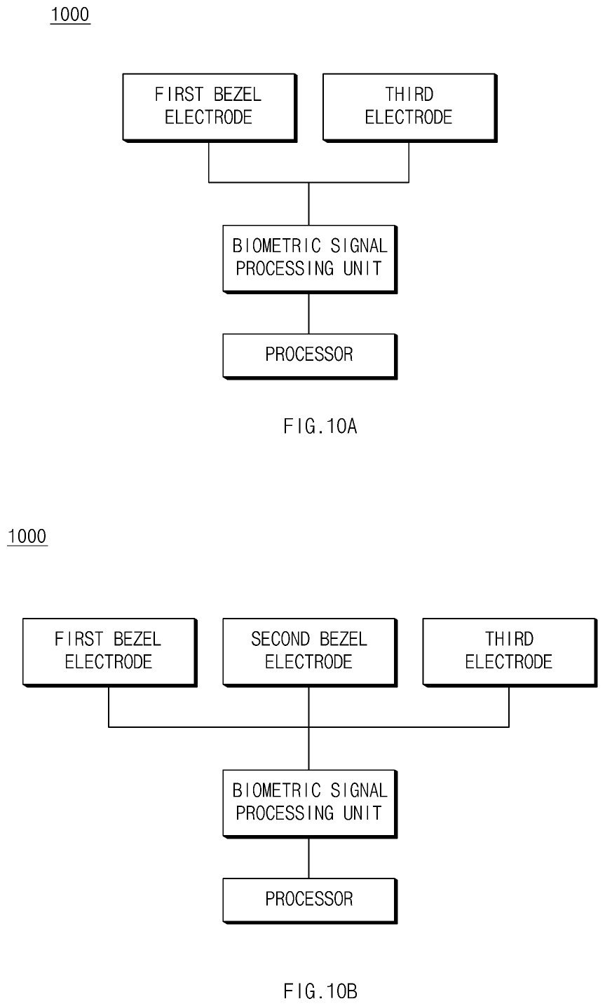

In various embodiments, the first bearing structure 250 may electrically connect the conductive portion (e.g., the conductive portion 221 or 222 of FIG. 4) of the wheel member 220 and the printed circuit board 240. For example, a biometric sensor (e.g., a biometric signal processing unit of FIGS. 10A and 10B) may be disposed on the printed circuit board 240, and the conductive portion (e.g., the conductive portion 221 or 222 of FIG. 4) of the wheel member 220 may be a biometric electrode that makes contact with a part of a user's body and obtains biometric information of the user. The first bearing structure 250 may electrically connect the biometric sensor and the biometric electrode. The biometric sensor may include, for example, an electrocardiography sensor.

In various embodiments, the electronic device 200 may include the plurality of first bearing structures 250. Each of the first bearing structures 250 may be electrically connected with one conductive portion (e.g., the first conductive portion 221 or the second conductive portion 222 of FIG. 4). Referring to FIG. 5, bearing structure 1-1 250-1 illustrated on a left side may be electrically connected with a first conductive portion (e.g., the first conductive portion 221 of FIG. 4) of the wheel member 220, and bearing structure 1-2 250-2 illustrated on a right side may be electrically connected with a second conductive portion (e.g., the second conductive portion 222 of FIG. 4) of the wheel member 220.

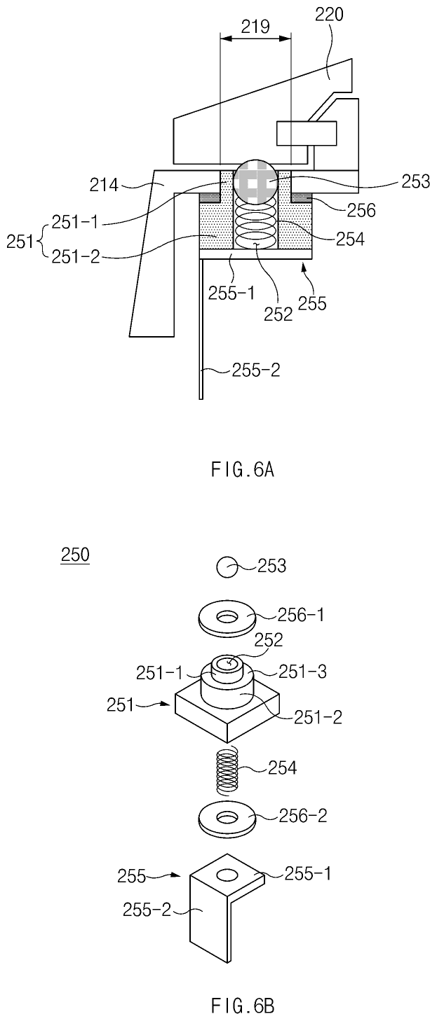

FIGS. 6A and 6B are diagrams illustrating an example bearing structure of the electronic device according to various embodiments.

Referring to FIGS. 6A and 6B, the first bearing structure 250 may support rotation of the wheel member 220. At least part of the first bearing structure 250 may form part of the surface of the front cover 214 through the through-hole 219. The first bearing structure 250 may include the bracket 251, the conductive ball 253, the elastic member 254, and the connecting member 255.

In an embodiment, at least part of the bracket 251 may be formed inside the through-hole 219. The bracket 251 may surround the elastic member 254 and part of the conductive ball 253. The bracket 251 may support rotation of the conductive ball 253. The waterproof member 256 may be coupled to the bracket 251. The waterproof member 256 may prevent and/or reduce infiltration of moisture into the front cover 214. The wheel member 220 may be disposed over the bracket 251, and the connecting member 255 may be disposed under the bracket 251.

In various embodiments, the bracket 251 may include the first portion 251-1 formed in a size corresponding to the through-hole 219, the second portion 251-2 formed to be larger than the through-hole 219, and the opening 252 formed through the first portion 251-1 and the second portion 251-2. A step surface 251-3 may be formed between the first portion 251-1 and the second portion 251-2. The waterproof member 256 may be disposed between the step surface 251-3 and the front cover 214.

In various embodiments, the opening 252 may be aligned with the through-hole 219. The conductive ball 253 and the elastic member 254 may be disposed in the opening 252. The opening 252 may be hidden by the connecting member 255.

In various embodiments, at least part of the conductive ball 253 may protrude from the surface of the front cover 214 such that the wheel member 220 and the front cover 214 are spaced apart from each other at a predetermined interval. Accordingly, the conductive ball 253 may decrease a frictional force between the wheel member 220 and the front cover 214 when the wheel member 220 rotates. In various embodiments, the conductive ball 253 may be rotated inside the opening 252 of the bracket 251 when the wheel member 220 rotates. The elastic member 254 may maintain the position of the conductive ball 253.

In an embodiment, the elastic member 254 may be formed to be conductive. The elastic member 254 may be electrically connected with the conductive ball 253. Furthermore, the elastic member 254 may apply an elastic force to the conductive ball 253 such that the conductive ball 253 makes contact with the wheel member 220. One side of the elastic member 254 may be connected to the connecting member 255, and an opposite side of the elastic member 254 may be connected to the conductive ball 253. The elastic member 254 may be surrounded by the bracket 251.

In various embodiments, the conductive ball 253, the elastic member 254, and the connecting member 255 may electrically connect a conductive portion (e.g., the first conductive portion 221 or the second conductive portion 222 of FIG. 4) of the wheel member 220 and a printed circuit board (e.g., the printed circuit board 240 of FIG. 4).

In an embodiment, the first bearing structure 250 may include at least one waterproof member 256. For example, the first bearing structure 250 may include a first waterproof member 256-1 formed between the front cover 214 and the step surface 251-3 of the bracket 251 and a second waterproof member 256-2 formed between the bracket 251 and the connecting member 255. The waterproof members 256-1 and 256-2 may prevent and/or reduce infiltration of moisture into the space inward of the front cover 214.

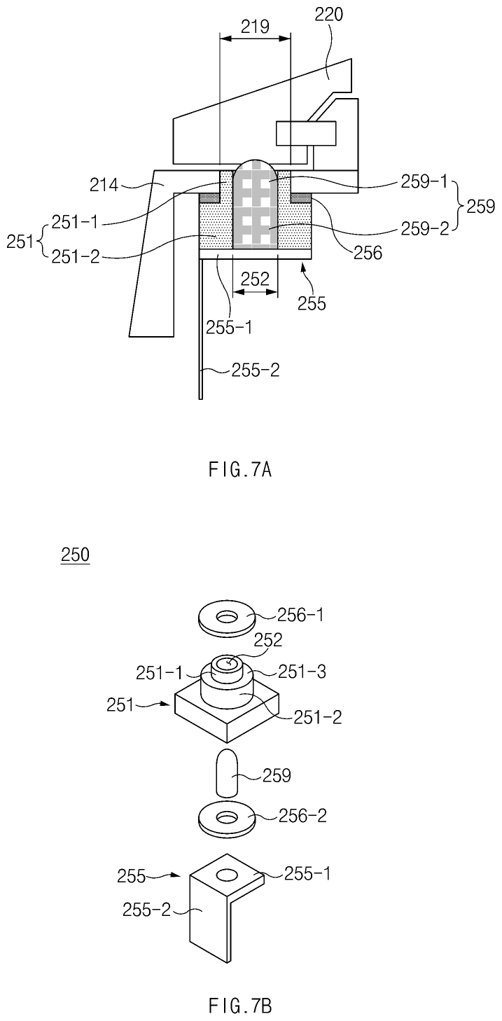

FIGS. 7A and 7B are diagrams illustrating an example bearing structure of the electronic device according to various embodiments.

In an embodiment, the first bearing structure 250 may include the bracket 251, a flexible structure (which may be referred to hereinafter as a bearing) 259, the waterproof member 256, and the connecting member 255.

In an embodiment, at least part of the bracket 251 may be disposed in the through-hole 219 formed in the front cover 214. The bracket 251 may include the first portion 251-1 formed in a size corresponding to the through-hole 219, the second portion 251-2 formed to be larger than the through-hole 219, and the opening 252 formed through the first portion 251-1 and the second portion 251-2. The step surface 251-3 may be formed between the first portion 251-1 and the second portion 251-2. The waterproof member 256 may be disposed between the step surface 251-3 and the front cover 214. Part of the flexible structure (bearing) 259 may be disposed in the opening 252 of the bracket 251. The support portion 255-1 of the connecting member 255 may be disposed on the second portion 251-2.

In an embodiment, the flexible structure 259 may be formed to be conductive. The flexible structure (bearing) 259 may electrically connect the conductive portion (e.g., the conductive portion 221 or 222 of FIG. 4) of the wheel member 220 and the connecting member 255. At least part of the flexible structure 259 may be disposed in the opening 252 of the bracket 251, and the remaining part may protrude from the surface of the front cover 214 toward the wheel member 220. The flexible structure 259 may include a protruding portion 259-1 protruding from the surface of the front cover 214 and a connecting portion 259-2 surrounded by the bracket 251 and electrically connected with the connecting member 255. The protruding portion 259-1 may be formed in a round shape. The protruding portion 259-1 may support rotation of the wheel member 220. The connecting portion 259-2 may be electrically connected with the connecting member 255.

In various embodiments, the flexible structure 259 may apply an elastic force to the wheel member 220 to support rotation of the wheel member 220. In various embodiments, the flexible structure 259 may be disposed such that the wheel member 220 is spaced apart from the surface of the front cover 214 at a predetermined interval. The flexible structure 259 may space the wheel member 220 apart from the front cover 214 to prevent and/or reduce the wheel member 220 from being directly rubbed against the front cover 214 when rotating. In various embodiments, the flexible structure 259 may apply an elastic force to the wheel member 220 such that the wheel member 220 is spaced apart from the surface of the front cover 214.

In an embodiment, the connecting member 255 may include the support portion 255-1 that covers the opening 252 of the bracket 251 and the extension portion 255-2 that extends from the support portion 251-1 to a printed circuit board (e.g., the printed circuit board 240 of FIG. 5). The support portion 255-1 may be electrically connected with the flexible structure 259 and the conductive portion (e.g., the conductive portion 221 or 222 of FIG. 4) of the wheel member 220 by making contact with the flexible structure 259. The connecting member 255 may include a flexible printed circuit board.

In an embodiment, the first bearing structure 250 may include at least one waterproof member 256. For example, the first bearing structure 250 may include the first waterproof member 256-1 formed between the front cover 214 and the step surface 251-3 of the bracket 251 and the second waterproof member 256-2 formed between the bracket 251 and the connecting member 255. The waterproof members 256-1 and 256-2 may prevent and/or reduce infiltration of moisture into the space inward of the front cover 214.

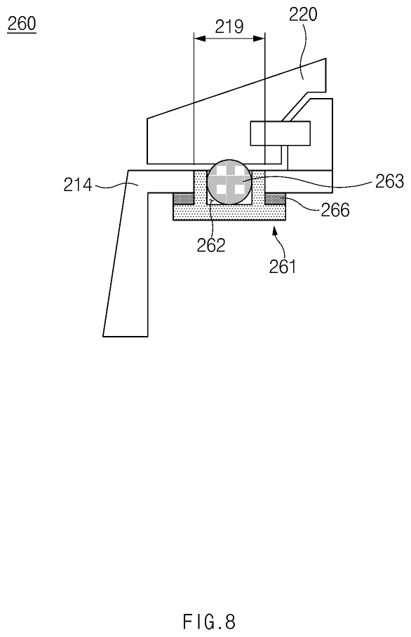

FIG. 8 is a diagram illustrating an example bearing structure of the electronic device according to various embodiments.

In an embodiment, the second bearing structures 260 may support rotation of the wheel member 220. Unlike the first bearing structures (e.g., the first bearing structures 250 of FIGS. 6A to 7B), the second bearing structure 260 may not provide electrical connection to the wheel member 220.

In an embodiment, the second bearing structure 260 may include a ball 263, a bracket 261 that supports the ball 263, and a waterproof member 266. At least part of the bracket 261 may be disposed in a through-hole 219 of the front cover 214. The bracket 261 may include an opening 262 in which the ball 263 is disposed. The ball 263 may be configured to rotate inside the opening 262 of the bracket 261 when the wheel member 220 rotates. The waterproof member 266 may be disposed between a step surface of the bracket 261 and an inner surface of the front cover 214. The waterproof member 266 may prevent and/or reduce infiltration of moisture between the through-hole 219 and the bracket 261.

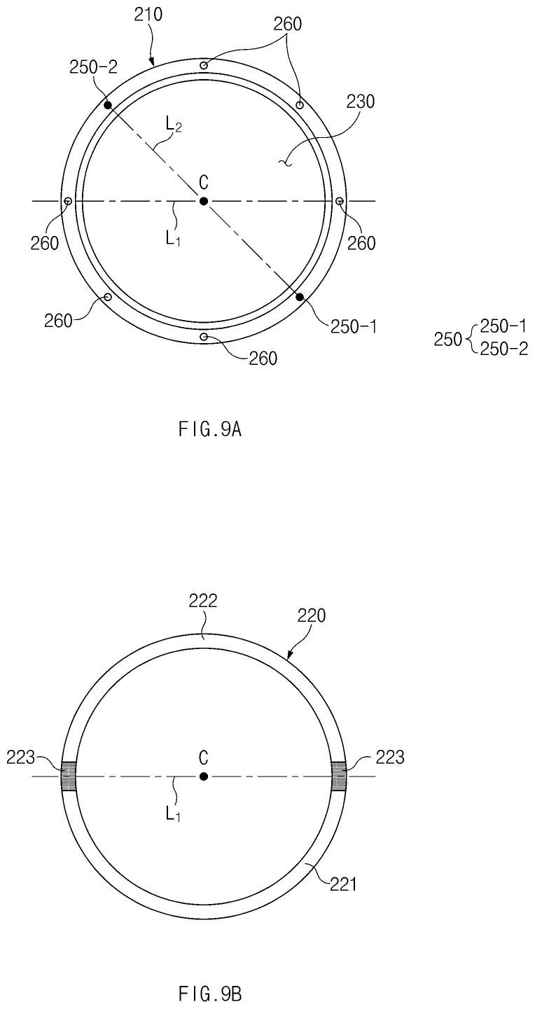

FIGS. 9A and 9B are diagrams illustrating example bearing structures and the wheel member of the electronic device according to various embodiments. FIGS. 9A and 9B are top views of the electronic device illustrated in FIGS. 5, 6A, 6B, 7A, 7B and 8.

In the illustrated embodiment, the bearing structures 250 and 260 may include the first bearing structures 250 that are electrically connected with the conductive portions 221 and 222 of the wheel member 220 and that support rotation of the wheel member 220, and the second bearing structures 260 that support rotation of the wheel member 220. Unlike the first bearing structures 250, the second bearing structures 260 may be insulated from the conductive portions 221 and 222 of the wheel member 220. The number of first bearing structures 250 may be equal to or larger than the number of conductive portions 221 and 222 of the wheel member 220. For example, the first bearing structures 250 may include bearing structure 1-1 250-1 connected with the first conductive portion 221 and bearing structure 1-2 250-2 connected with the second conductive portion 222.

In the illustrated embodiment, the wheel member 220 may include the first conductive portion 221, the second conductive portion 222, and the insulating portions 223 that insulate the first conductive portion 221 and the second conductive portion 222. The first conductive portion 221 may be electrically connected with bearing structure 1-1 250-1. The second conductive portion 222 may be electrically connected with bearing structure 1-2 250-2. The conductive portions 221 and 222 may be electrically connected with the first bearing structures 250 by making contact with the protruding portions (e.g., the conductive ball 253 of FIGS. 6A and 6B or the flexible structure 259 of FIGS. 7A and 7B) of the first bearing structures 250.

In various embodiments, bearing structure 1-1 250-1 and bearing structure 1-2 250-2 may make contact with the conductive portions 221 and 222 on opposite sides of a virtual line L1 that connects the insulating portions 223 of the wheel member 220. Accordingly, electrical connection between the first conductive portion 221 and the second conductive portion 222 may be prevented. For example, bearing structure 1-1 250-1 may make contact with a lower part of the wheel member 220 with respect to the virtual line L1 illustrated in FIGS. 9A and 9B, and bearing structure 1-2 250-2 may make contact with an upper part of the wheel member 220. In various embodiments, the virtual line L1 may be a line that passes through the center C of rotation of the wheel member 220.

In various embodiments, bearing structure 1-1 250-1 and bearing structure 1-2 250-2 may be formed in positions opposite to each other. For example, bearing structure 1-1 250-1 and bearing structure 1-2 250-2 may be disposed such that a virtual line L2 connecting bearing structure 1-1 250-1 and bearing structure 1-2 250-2 passes through the center C of rotation of the wheel member 220. For example, bearing structure 1-1 250-1 and bearing structure 1-2 250-2 may be circumferentially spaced apart from each other by 180 degrees.

In various embodiments, the bearing structures (the first bearing structures 250 and the second bearing structures 260) may be circumferentially arranged at predetermined intervals. For example, any one bearing structure may be circumferentially spaced apart from other bearing structures adjacent thereto by the same angle. Accordingly, the bearing structures 250 and 260 may stably support rotation of the wheel member 220.

In various embodiments, the wheel member 220 may rotate in a range in which bearing structure 1-1 250-1 maintains contact with the first conductive portion 221 and bearing structure 1-2 250-2 maintains contact with the second conductive portion 222. For example, the wheel member 220 may rotate from a state in which the insulating portion 223 is adjacent to one side of bearing structure 1-1 250-1 to a state in which the insulating portion 223 is adjacent to an opposite side of bearing structure 1-2 250-2. That is, the wheel member 220 may rotate in a range in which the insulating portions 223 do not make contact with the first bearing structures 250-1 and 250-2.

In various embodiments, a part (e.g., a finger) of the user's body may be brought into contact with the first conductive portion 221 and the second conductive portion 222. Predetermined electrical signals may be applied to the conductive portions 221 and 222 from the part of the user's body. For example, a first biometric signal may be applied to the first conductive portion 221, and a second biometric signal may be applied to the second conductive portion 222.

In various embodiments, the first bearing structures 250 may include bearing structure 1-1 250-1 through which the first biometric signal flows and bearing structure 1-2 250-2 through which the second biometric signal flows.

In various embodiments, the first biometric signal may be applied to at least one of the plurality of first bearing structures 250, and the second biometric signal may be applied to at least one other first bearing structure.

In various embodiments, depending on the rotation angle of the wheel member 220, one of the plurality of first bearing structures 250 may be bearing structure 1-1 250-1 through which the first biometric signal flows or bearing structure 1-2 250-2 through which the second biometric signal flows.

For example, in a first state in which the wheel member 220 rotates through a predetermined angle, any one first bearing structure 250 may make contact with the first conductive portion 221 and may function as bearing structure 1-1 250-1. After the wheel member 220 additionally rotates through a predetermined angle in the first state, the first bearing structure 250 may make contact with the second conductive portion 222 and may function as bearing structure 1-2 250-2.

In some embodiments, the wheel member 220 may rotate without a limitation in rotation range. For example, the first biometric signal may be applied to some of the plurality of first bearing structures 250, and the second biometric signal may be applied to the other first bearing structures. That is, when the plurality of first bearing structures 250-1 and 250-2 are electrically connected to the conductive portions 221 and 222, respectively, the wheel member 220 may rotate without a limitation in rotation range.

In various embodiments, irrespective of the rotation angle of the wheel member 220, the first biometric signal may be applied to one (e.g., bearing structure 1-1 250-1) of the two first bearing structures 250, and the second biometric signal may be applied to the other (e.g., bearing structure 1-2 250-2). That is, two distinct biometric signals may be applied to the first bearing structures 250. For example, the first biometric signal may be applied to the first bearing structure 250 located on one side of the virtual line L1, and the second biometric signal may be applied to the first bearing structure 250 located on an opposite side of the virtual line L1.

FIGS. 10A and 10B are block diagrams illustrating example biometric signal acquisition systems of an electronic device according to various embodiments.

Referring to FIGS. 10A and 10B, the electronic device 1000 may further include a biometric signal processing unit (e.g., including various circuitry) for detecting a biometric signal of a user and a processor (e.g., including processing circuitry). The biometric signal processing unit may include a biometric sensor that senses the user's biometric signal. The biometric sensor and the processor may be mounted on a printed circuit board (e.g., the printed circuit board 240 of FIG. 5) that is disposed inside the electronic device 1000. In various embodiments, the user's biometric signal may include an electrocardiogram signal.

In various embodiments, the biometric signal processing unit may be electrically connected with at least two electrodes (e.g., at least two of a first bezel electrode, a second bezel electrode, and a third electrode). The at least two electrodes may be exposed on a surface (e.g., the first surface 110A, the second surface 110B, or the side surfaces 110C of FIGS. 1 and 2) of the electronic device 1000 and may make contact with a part (e.g., a finger) of the user's body.

In various embodiments, the at least two electrodes may include a bezel electrode (e.g., the conductive portion 221 or 222 of FIGS. 4, 9A, and 9B) that is formed in a wheel member (e.g., the wheel member 220 of FIGS. 4, 9A, and 9B) of the electronic device 1000 and/or the third electrode formed on a surface (e.g., the back plate 107 of FIG. 2) of a housing of the electronic device 1000.

In various embodiments, the processor may be configured to detect biometric information of the user, based on the user's biometric signal sensed by the biometric signal processing unit.

An electronic device according to various example embodiments of the disclosure may include: a housing having at least one through-hole formed in a surface thereof, a rotatable wheel coupled to the surface of the housing including a conductive portion, a printed circuit board disposed in the housing, and a first bearing structure comprising a bearing, at least part of which is disposed in the through-hole to support rotation of the wheel, wherein the first bearing structure is configured to electrically connect the printed circuit board and the conductive portion.

In various example embodiments, the first bearing structure may include a conductive ball contacting the conductive portion of the wheel, and the conductive ball may protrude from the surface of the housing.

In various example embodiments, the first bearing structure may include a connecting member including a conductive material extending to the printed circuit board, and the connecting member may include a flexible printed circuit board.

In various example embodiments, the first bearing structure may include a conductive ball, at least part of which protrudes from the surface of the housing, a connecting member disposed in the housing and extending to the printed circuit board, and an elastic member comprising a conductive material disposed between the conductive ball and the connecting member.

In various example embodiments, the first bearing structure may further include a bracket disposed in the housing, at least part of the bracket extending into the through-hole, and the bracket may include an opening in which the conductive ball and the elastic member are disposed.

In various example embodiments, the conductive ball may be configured to roll in the opening as the wheel member rotates.

In various example embodiments, the bracket may include a first portion having a size corresponding to the through-hole, a second portion having a size larger than the through-hole, and a step surface between the first portion and the second portion, and the conductive ball may be disposed in the first portion.

In various example embodiments, the connecting member may include a support portion disposed on the second portion to cover the opening and an extension portion extending from the support portion to the printed circuit board.

In various example embodiments, the first bearing structure may further include a waterproof member comprising a waterproofing material disposed between the step surface and an inner surface of the housing.

In various example embodiments, the number of first bearing structures may be equal to or greater than the number of conductive portions.

In various example embodiments, the first bearing structure may include a conductive structure comprising a conductive material extending toward the wheel member through the through-hole, at least part of the conductive member being disposed in the housing, and the conductive structure may be formed to be flexible.

In various example embodiments, the first bearing structure may further include a connecting member comprising a conductive material extending from the conductive structure to the printed circuit board.

In various example embodiments, the electronic device may further include a second bearing structure configured to support rotation of the wheel, and the second bearing structure may include a ball, at least part of which is disposed in the through-hole.

In various example embodiments, the ball may be non-conductive.

In various embodiments, one of the first bearing structure and the second bearing structure may be arranged at a same interval as another bearing structure adjacent thereto.

In various example embodiments, the wheel may include a first conductive portion, a second conductive portion electrically insulated from the first conductive portion, and an insulating portion disposed between the first conductive portion and the second conductive portion, and the first bearing structure may include a first bearing structure contacting the first conductive portion and a second first bearing structure contacting the second conductive portion.

In various example embodiments, the electronic device may further include a biometric sensor configured to sense a biometric signal and a processor configured to detect biometric information, based on the biometric signal, and the biometric sensor may be electrically connected with the first bearing structure.

In various example embodiments, the electronic device may further include at least two electrodes electrically connected to the biometric sensor and the processor, and the at least two electrodes may include the conductive portion.

In various example embodiments, the at least two electrodes may further include an electrode provided on the surface of the housing.

In various example embodiments, the biometric information may include information related to an electrocardiogram.

According to the various example embodiments of the disclosure, an electronic device capable of measuring an electrocardiogram of a user by a touch of a part of the user's body on a wheel member is provided.

According to the various example embodiments of the disclosure, an electronic device that includes at least two electrodes formed in a wheel member and that is capable of measuring body fat through the electrodes is provided.

In addition, the disclosure may provide various effects that are directly or indirectly recognized.

The electronic device according to various embodiments may be one of various types of electronic devices. The electronic devices may include, for example, a portable communication device (e.g., a smartphone), a computer device, a portable multimedia device, a portable medical device, a camera, a wearable device, a home appliance, or the like. According to an embodiment of the disclosure, the electronic devices are not limited to those described above.

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B”, “at least one of A and B”, “at least one of A or B”, “A, B, or C”, “at least one of A, B, and C”, and “at least one of A, B, or C” may include any one of, or all possible combinations of the items enumerated together in a corresponding one of the phrases. As used herein, such terms as “1st” and “2nd”, or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with”, “coupled to”, “connected with”, or “connected to” another element (e.g., a second element), the element may be coupled with the other element directly (e.g., wiredly), wirelessly, or via a third element.

As used herein, the term “module” may include a unit implemented in hardware, software, or firmware, or any combination thereof, and may interchangeably be used with other terms, for example, “logic”, “logic block”, “part”, or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., a program) including one or more instructions that are stored in a storage medium (e.g., internal memory or external memory) that is readable by a machine (e.g., the electronic device). For example, a processor (e.g., the processor) of the machine (e.g., the electronic device) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a compiler or a code executable by an interpreter. The machine-readable storage medium may be provided in the form of a non-transitory storage medium. Wherein, the “non-transitory” storage medium is a tangible device, and may not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

While the disclosure has been illustrated and described with reference to various example embodiments, it will be understood that the various example embodiments are intended to be illustrative, not limiting. It will be further understood by those skilled in the art that various changes in form and detail may be made therein without departing from the true spirit and full scope of the disclosure, including the appended claims and their equivalents.

What is claimed is:

1. An electronic device comprising:
a housing having at least one through-hole in a surface thereof;
a rotatable wheel coupled to the surface of the housing, the wheel including a conductive portion;
a printed circuit board disposed in the housing; and
a first bearing structure including a bearing, at least part of which is disposed in the through-hole and configured to support rotation of the wheel, wherein the first bearing structure electrically connects the printed circuit board and the conductive portion.

2. The electronic device of claim 1, wherein the first bearing structure includes a conductive ball contacting the conductive portion of the wheel, and
wherein the conductive ball protrudes from the surface of the housing.

3. The electronic device of claim 1, wherein the first bearing structure includes a connecting member comprising a conductive material extending to the printed circuit board, and
wherein the connecting member comprises a flexible printed circuit board.

4. The electronic device of claim 1, wherein the first bearing structure includes a conductive ball, at least part of which protrudes from the surface of the housing, a connecting member comprising a conductive material disposed in the housing and extending to the printed circuit board, and an elastic member comprising a conductive material disposed between the conductive ball and the connecting member.

5. The electronic device of claim 4, wherein the first bearing structure further includes a bracket disposed in the housing, at least part of the bracket extending into the through-hole, and
wherein the bracket includes an opening in which the conductive ball and the elastic member are disposed.

6. The electronic device of claim 5, wherein the conductive ball is configured to roll in the opening as the wheel rotates.

7. The electronic device of claim 5, wherein the bracket includes a first portion having a size corresponding to the through-hole, a second portion having a size larger than the through-hole, and a step surface between the first portion and the second portion, and
wherein the conductive ball is disposed in the first portion.

8. The electronic device of claim 7, wherein the connecting member includes a support portion disposed on the second portion to cover the opening and an extension portion extending from the support portion to the printed circuit board.

9. The electronic device of claim 7, wherein the first bearing structure further includes a waterproof member comprising a waterproofing material disposed between the step surface and an inner surface of the housing.

10. The electronic device of claim 1, wherein a number of first bearing structures is equal to or greater than a number of conductive portions.

11. The electronic device of claim 1, wherein the first bearing structure includes a conductive structure comprising a conductive material extending toward the wheel through the through-hole, at least part of the conductive member being disposed in the housing, and
wherein the conductive structure is flexible.

12. The electronic device of claim 11, wherein the first bearing structure further includes a connecting member comprising a conductive material extending from the conductive structure to the printed circuit board.

13. The electronic device of claim 1, further comprising:
a second bearing structure configured to support rotation of the wheel,
wherein the second bearing structure includes a ball, at least part of which is disposed in the through-hole.

14. The electronic device of claim 13, wherein the ball of the second bearing structure is non-conductive.

15. The electronic device of claim 13, wherein one of the first bearing structure and the second bearing structure is arranged at a same interval as another bearing structure adjacent thereto.

16. The electronic device of claim 1, wherein the wheel includes a first conductive portion, a second conductive portion electrically insulated from the first conductive portion, and an insulating portion disposed between the first conductive portion and the second conductive portion, and
wherein the first bearing structure includes a first bearing structure contacting the first conductive portion and a second first bearing structure contacting the second conductive portion.

17. The electronic device of claim 1, further comprising:
a biometric sensor configured to sense a biometric signal; and
a processor configured to detect biometric information, based on the biometric signal,
wherein the biometric sensor is electrically connected with the first bearing structure.

18. The electronic device of claim 17, wherein the electronic device further comprises at least two electrodes electrically connected with the biometric sensor and the processor, and
wherein the at least two electrodes include the conductive portion.

19. The electronic device of claim 18, wherein the at least two electrodes further include an electrode on the surface of the housing.

20. The electronic device of claim 17, wherein the biometric information includes information related to an electrocardiogram.
